(19) United States
(12) Patent Application Publication
(10) Pub. No.: US 2022/0168343 A1
(43) Pub. Date: Jun. 2, 2022

(54) CANCER IMMUNOTHERAPY USING COMBINATIONS OF CELLS EXPRESSING CHIMERIC ANTIGEN RECEPTORS AND MONOCLONAL ANTIBODIES

(71) Applicants: CoImmune, Inc., Durham, NC (US); FONDAZIONE MATILDE TETTAMANTI E MENOTTI DE MARCHI ONLUS, Monza (IT)

(72) Inventors: Maurits W. GEERLINGS, Berwyn, PA (US); Andrea BIONDI, Monza (IT); Alessandro RAMBALDI, Bergamo (IT); Giuseppe DASTOLI, Monza (IT)

(21) Appl. No.: 17/430,540

(22) PCT Filed: Feb. 13, 2020
(86) PCT No.: PCT/US2020/018127

§ 371 Date: Aug. 12, 2021

Related U.S. Application Data
(60) Provisional application No. 62/805,052, filed on Feb. 13, 2019.

(51) Int. Cl. A61K 35/17; A61K 38/17; A61P 35/00; A61K 47/68

(52) U.S. Cl. CPC A61K 35/17; A61K 38/1774; A61K 2039/505; A61K 47/6809; A61K 47/6849; A61P 35/00

(57) ABSTRACT

Methods of increasing or enhancing the efficacy of CAR B cell malignancy treatment regimens using immune effector cells (e.g., T cells, NK cells, CIK cells, macrophages) engineered to express chimeric antigen receptors (CAR(s)) that target malignant B cells in combination with antibodies (e.g., monoclonal antibodies, antibody-drug conjugates) that target malignant B cells are provided. Also provided are methods of treating a B cell malignancy in a subject comprising administering to the subject a CAR B cell malignancy treatment regimen and an antibody that targets malignant B cells.

### CROSS-REFERENCE TO RELATED APPLICATIONS

This application claims the benefit of U.S. Provisional Patent Application No. 62/805,052 filed Feb. 13, 2019 entitled “Cancer Immunotherapy Using Combinations of Cells Expressing Chimeric Antigen Receptors and Monoclonal Antibodies”, which is incorporated by reference herein in its entirety and for all purposes.

### FIELD OF THE INVENTION

The present disclosure relates generally to the use of immune effector cells (e.g., T cells, NK cells, CIK cells, macrophages) engineered to express chimeric antigen receptors (CAR(s)) that target malignant B cells in combination with antibodies (e.g., monoclonal antibodies, antibody-drug conjugates) that target malignant B cells to treat B cell malignancies.

### BACKGROUND OF THE INVENTION

Immunotherapy is a promising approach for the treatment of cancer. Immunotherapy with cells expressing chimeric antigen receptors (CARs) that target antigens expressed by the tumor has the advantage of targeted therapies that can invoke a rapid and sustained immune response against a cancer. CAR therapy has shown promising results in the clinic in treating some hematological cancers, such as B cell malignancies (see, e.g., Novartis (2017) Prescribing Information for FDA approved products Kymriah™ and Yescarta™ incorporated by reference herein in their entirety). For an overview of CAR constructs, CAR therapy and CAR toxicities, see, X. Han, et al., Chronic Diseases and Translational Medicine 4 (2018) 225-243; and Tariq S, Haider S Ali, Hasan M, et al. (Oct. 23, 2018) Chimeric Antigen Receptor T-Cell Therapy: A Beacon of Hope in the Fight Against Cancer. Cureus 10(10). Combinations of CAR-T cells with antibodies that block the cytotoxic T-lymphocyte-associated antigen 4 (CTLA-4) or the programmed death-1 (PD-1) receptor or the PD-L1 ligand has been suggested to prolong the effector function of CAR-T cells at sites of solid tumors (Gianpietro Dotti, Stephen Gottschalk, Barbara Savoldo and Malcolm K, Brenner Immunol Rev. 2014 January; 257(1); and X U, et al., Oncology Letters 16: 2063-2070, 2018).

However, there exists a need for therapies that enhance the efficacy of CAR therapies and/or to treat B cell malignancies, as a significant number of patients receiving CAR therapies either relapse or remain refractory following such therapies.

### BRIEF SUMMARY OF THE INVENTION

The present invention is based, at least in part, upon an unexpected and surprising clinical outcome resulting from the consecutive administration of two therapeutic drugs, each having distinctive mechanisms of action. The first drug regarded a chimeric antigen receptor-modified T cell immunotherapy. The second drug regarded an antibody-drug conjugate used to treat relapsed or refractory B-cell precursor acute lymphoblastic leukemia (see Example section for further details).

Accordingly, the present invention concerns, at least in part, methods for treating a disease associated with expression of a tumor antigen, for example, a B cell malignancy, in a subject by administering to the subject a combination of a CAR immunotherapy and an antibody, for example an antibody-drug conjugate.

In one aspect, the invention includes a method of increasing or enhancing the efficacy of a CAR B cell malignancy treatment regimen in a subject comprising administering to the subject a CAR immunotherapy that targets malignant B cells and an antibody that targets malignant B cells.

In another aspect the invention includes a method of treating a B cell malignancy in a subject comprising administering to the subject a CAR B cell malignancy treatment regimen and an antibody that targets malignant B cells.

In other embodiments of any of the methods described herein, the antigen binding domain of the CAR molecule targets (e.g., binds to) a tumor antigen that is associated with a B cell malignancy, e.g., expressed by a malignant B cell. In some embodiments, the tumor antigen is present in a disease chosen from a leukemia or a lymphoma.

### DETAILED DESCRIPTION OF THE INVENTION

### Definitions

Unless otherwise defined, all technical and scientific terms used herein have the same meaning as commonly understood by one of ordinary skill in the art to which this invention pertains. Although methods and materials similar or equivalent to those described herein can be used in the practice of the present invention, suitable methods and materials are described below. All publications, patent applications, patents, and other references mentioned herein are expressly incorporated by reference in their entirety. In cases of conflict, the present specification, including definitions, will control. In addition, the materials, methods, and examples described herein are illustrative only and are not intended to be limiting.

As used herein, the singular form “a” “an”, and “the” include plural referents unless the context clearly dictates otherwise. For example, reference to “a cell” includes a plurality of such cells and reference to “an antibody” includes a plurality of such antibody.

The term “B cell antigen” refers to a molecule that is preferentially expressed on the surface of a B cell which can be targeted with an agent which binds thereto. The B cell antigen of particular interest is preferentially expressed on malignant B cells compared to other non-malignant B cells or non-B cells of a mammal. Examples of B cell antigens include CD10, CD19, CD20, CD21, CD22, CD23, CD24, CD25, CD34, CD37, CD38, CD53, CD72, CD73, CD74, CD75, CD77, CD79a, CD79b, CD80, CD81, CD82, CD83, CD84, CD85, CD86, CD123, CD179b, ROR1, BCMA, and FLT3.

As used herein, a “B cell malignancy” refers to all types of B cell malignancies found in mammals and known in the art, including, but not limited to solid tumors and hematological cancers.

As used herein, “hematological cancer” refers to all types of hematological cancer and hematopoietic tumors, neoplasm or malignant tumors found in mammals, including, but not limited to leukemias and lymphomas. Examples include, Hodgkin's Disease, Non-Hodgkin's Lymphoma, B-cell Acute Lymphoid Leukemia (“B-ALL”), T-cell Acute Lymphoid Leukemia (“T-ALL”), Acute Lymphoblastic Leukemia (ALL); one or more chronic leukemias including but not limited to, e.g., Chronic Myelogenous Leukemia (CML), Chronic Lymphoid Leukemia (CLL), or other hematological malignancies.

Administered “in combination”, as used herein, means that two (or more) different treatments are administered to the subject during the course of the subject's affliction with the disorder, e.g., the two or more treatments are administered after the subject has been diagnosed with the disorder and before the disorder has been cured or eliminated or treatment has ceased for other reasons. In some embodiments, the administration of one treatment is still occurring when the administration of the second begins, so that there is overlap in terms of administration. This is sometimes referred to herein as “simultaneous” or “concurrent” administration. In other embodiments, the administration of one treatment ends before the administration of the other treatment begins. In some embodiments of either case, the treatment is more effective because of combined administration and results in an unexpectedly superior effect compared to the effect obtained with the individual treatments. For example, the first or second treatment is more effective, e.g., an increased or enhanced effect of the first treatment is seen after the second treatment or an equivalent effect is seen with less of the first treatment, than would be seen if the first treatment were administered in the absence of the second treatment, or the analogous situation is seen with the second treatment. In some embodiments, administration is such that the reduction in a symptom, or other parameter related to the disorder is greater than what would be observed with one treatment administered in the absence of the other. The effect of the two treatments can be partially additive, wholly additive, or greater than additive (i.e., synergistic). The administration can be such that an effect of the first treatment administered is still detectable when the second is administered.

The term “Chimeric Antigen Receptor” or alternatively “CAR(s)” refers to a recombinant polypeptide construct comprising at least an extracellular antigen binding domain, a transmembrane domain and a cytoplasmic signaling domain (also referred to herein as an “intracellular signaling domain,” a cytoplasmic signaling domain” or a “stimulatory molecule”) comprising a functional signaling domain derived from a stimulatory molecule. The terms “Chimeric Antigen Receptor” and “CAR” include CARs that are generally known in the art (see, e.g., Shi et al., Molecular Cancer 2014, 13:219, which is incorporated herein in its entirety).

The cytoplasmic signaling domain can comprise a primary signaling domain (e.g., a primary signaling domain of CD3-zeta). The cytoplasmic signaling domain can further comprise one or more functional signaling domains derived from at least one costimulatory molecule. The costimulatory molecule is chosen from 4-1BB (i.e., CD137), CD27, ICOS, and/or CD28.

A CAR that comprises an antigen binding domain (e.g., a single chain variable fragment of a monoclonal antibody (“scFv”)) that targets, e.g., binds to, a specific antigen X, such as those described herein, is also referred to as X CAR. For example, a CAR that comprises an antigen binding domain that targets CD19 is referred to as CD19 CAR. A CAR that comprises an antigen binding domain (e.g., a scFv) that targets a specific tumor antigen (TA) is also referred to as TA CAR. A CAR that comprises an antigen binding domain (e.g., a scFv) that targets a specific B cell antigen (BCA) is also referred to as BCA CAR.

The terms “CAR immunotherapy cancer treatment that targets malignant B cells” and “CAR B cell malignancy treatment regimen” and the like refer to treatment of a subject with immune cells (e.g., T cells, NK cells, CIK cells, macrophages) that have been genetically engineered to express chimeric antigen receptors (CARs) and specifically target one or more tumor-specific receptors of malignant B cells. CAR immunotherapy is generally known in the art (see, e.g., Shi et al., Molecular Cancer 2014, 13:219, which is incorporated herein in its entirety).

The CAR construct can be introduced into immune effector cells (e.g., T cells, NK cells, CIK cells, macrophages) using viral or non-viral techniques known in the art.

The term “antibody,” as used herein, refers to a protein, or polypeptide sequence derived from an immunoglobulin molecule which specifically binds with an antigen. Antibodies can be polyclonal or monoclonal, multiple or single chain, or intact immunoglobulins, and may be derived from natural sources or from recombinant sources. Antibodies can be tetramers of immunoglobulin molecules. The term “antibody” includes functional antibody fragments, including e.g., Fab′, F(ab′)2, Fab, Fv, and scFv fragments. Antibodies can be humanized, human, and/or antibody drug-conjugates. The term “antibody” includes antibodies that are generally known in the art.

As used herein, the term “effective amount,” “safe and effective amount” or “therapeutically effective amount” and the like refers to the quantity of a component which is sufficient to yield a desired therapeutic response without undue adverse side effects (such as toxicity, irritation, or allergic response) commensurate with a reasonable benefit/risk ratio when used in the manner of this invention. For example, an amount effective to increase or enhance the efficacy of a treatment, delay the growth of or to cause a cancer to shrink or reduce malignant cell count in peripheral blood, bone marrow and/or other organs. The specific safe and effective amount or therapeutically effective amount will vary with such factors as the particular condition being treated, the physical condition of the patient, the type of mammal or animal being treated, the duration of the treatment, the nature of concurrent therapy (if any), and the specific formulations employed and the structure of the compounds (i.e., immune effector cells and/or antibodies).

As used herein, the term “enhancing the effect” or “increasing the effect” refers to reducing the population of cancer cells. The quantity, number, amount or percentage of cancer cells can be reduced by at least 25%, at least 30%, at least 40%, at least 50%, at least 65%, at least 75%, at least 85%, at least 95%, or at least 99% relative to a negative control.

As used herein, the terms “treat”, “treatment”, and “treating” refer to the reduction or amelioration of the progression, severity and/or duration of a cancer or proliferative disorder, or the amelioration of one or more symptoms (preferably, one or more discernible symptoms) of a cancer or proliferative disorder resulting from the administration of one or more therapies (e.g., one or more therapeutic agents such as a CAR and antibody of the invention). In specific embodiments, the terms “treat,” “treatment” and “treating” refer to the amelioration of at least one measurable physical parameter of a proliferative disorder, such as growth of a tumor, not necessarily discernible by the patient. In other embodiments the terms “treat”, “treatment” and “treating” refer to the inhibition of the progression of a cancer or proliferative disorder, either physically by, e.g., stabilization of a discernible symptom, physiologically by, e.g., stabilization of a physical parameter, or both. In other embodiments the terms “treat”, “treatment” and “treating” refer to the reduction or stabilization of tumor size or cancerous cell count.

Thus, treating may include enhancing or increasing efficacy, suppressing, inhibiting, preventing, treating, or a combination thereof. Treating refers inter alia to increasing time to disease progression, expediting remission, inducing remission, augmenting remission, speeding recovery, increasing efficacy of or decreasing resistance to alternative therapeutics, or a combination thereof. “Suppressing” or “inhibiting”, refers inter alia to delaying the onset of tumor associated symptoms, preventing relapse to a disease, decreasing the number or frequency of relapse episodes, increasing latency between symptomatic episodes, reducing the severity of symptoms, reducing the severity of an acute episode, reducing the number of symptoms, reducing the incidence of disease-related symptoms, reducing the latency of symptoms, ameliorating symptoms, reducing secondary symptoms, prolonging patient survival, or a combination thereof. The symptoms can be primary or secondary. “Primary” refers to a symptom that is a direct result of the proliferative disorder (e.g., cancer), while, secondary refers to a symptom that is derived from or consequent to a primary cause.

The term “subject” is intended to include living organisms (e.g., mammals, human).

“Relapsed” or “relapse” as used herein refers to the return or reappearance of a disease (e.g., cancer) or the signs and symptoms of a disease such as cancer after a period of improvement or responsiveness, e.g., after prior treatment of a therapy, e.g., cancer therapy. The initial period of responsiveness may involve the level of cancer cells falling below a certain threshold, e.g., below 20%, 10%, 5%, 4%, 3%, 2%, or 1%. The reappearance may involve the level of cancer cells rising above a certain threshold, e.g., above 20%, 1%, 10%, 5%, 4%, 3%, 2%, or 1%. For example, in the context of ALL, the reappearance may involve, e.g., a reappearance of blasts in the blood, bone marrow (>5%), or any extramedullary site, after a complete response. A complete response, in this context, may involve <5% bone marrow blasts. More generally, in an embodiment, a response (e.g., complete response or partial response) can involve the absence of detectable MRD (minimal residual disease). In an embodiment, the initial period of responsiveness lasts at least 1, 2, 3, 4, 5, or 6 days; at least 1, 2, 3, or 4 weeks; at least 1, 2, 3, 4, 6, 8, 10, or 12 months; or at least 1, 2, 3, 4, or 5 years.

### Description

The present invention is based in part upon the surprising discovery that a CAR immunotherapy cancer treatment regimen that targets malignant B cells in combination with administration of an antibody that targets B cells resulted in a greater therapeutic effect compared to the use of the CAR treatment alone.

Accordingly, the present invention provides methods of increasing or enhancing the efficacy of a CAR immunotherapy cancer treatment that targets malignant B cells in a subject in need thereof comprising administering to the subject an antibody that targets malignant B cells in combination with the CAR immunotherapy. Preferably, the cancer is a B cell malignancy. More preferably the B cell malignancy is a hematological cancer. Preferably, the CAR immunotherapy comprises immune effector cells (e.g., T cells, NK cells, CIK cells, macrophages) engineered to express a TA CAR or a BCA CAR. Preferably, the immune effector cells target a CD19 tumor antigen. Preferably, the antibody that targets malignant B cells is a humanized or human monoclonal antibody. Preferably the antibody that targets malignant B cells is a humanized or human monoclonal antibody that targets a CD22 tumor antigen.

Additionally, the present invention includes a method of treating a cancer in a subject in need thereof comprising administering to the subject an antibody that targets malignant B cells in combination with a CAR immunotherapy cancer treatment that targets malignant B cells. Preferably the cancer is a B cell malignancy. More preferably, the B cell malignancy is a hematologic cancer. Preferably, the CAR immunotherapy comprises immune effector cells (e.g., T cells, NK cells, CIK cells, macrophages) engineered to express a TA CAR or a BCA CAR. Preferably, the immune effector cells target a CD19 tumor antigen. Preferably, the antibody that targets malignant B cells is a humanized or human monoclonal antibody. Preferably the antibody that targets malignant B cells is a humanized or human monoclonal antibody that targets a CD22 tumor antigen.

The present invention also provides methods of reducing or eliminating existing tumor burden, reducing tumor volume, stimulating tumor regression, preventing relapse or increasing overall survival in a subject in need thereof comprising administering to the subject an antibody that targets malignant B cells in combination with a CAR immunotherapy cancer treatment that targets malignant B cells.

In embodiments wherein the immune effector cells are administered to a subject in combination with an antibody that targets malignant B cells, the subject may achieve one or more of the following: 1) increased tolerance to the immune effector cells; 2) increased efficacy of the immune effector cells; 3) reduced likelihood of rejection of the immune effector cells; and/or 4) increased or reduced adverse response that may be caused by the immune effector cells. Thus, the methods provided herein feature methods that result in increasing or enhancing the therapeutic efficacy of the immune effector cell therapy and/or the antibody therapy for treating a disease associated with the expression of a tumor antigen, e.g., a cancer described herein.

### Immune Effector Cells

In one embodiment, the present invention provides immune effector cells (e.g., T cells, NK cells, CIK cells, macrophages) that are engineered to contain one or more CARs that direct the immune effector cells to a malignant B cell. This is achieved through an antigen binding domain on the CAR that is specific for a malignant B cell antigen.

In an embodiment, the B cell antigen is an antigen that is expressed on the surface of the malignant B cell. The antigen can be expressed on the surface of any one of the following types of B cells: progenitor B cells (e.g., pre-B cells or pro-B cells), early pro-B cells, late pro-B cells, large pre-B cells, small pre-B cells, immature B cells, e.g., naive B cells, mature B cells, plasma B cells, plasmablasts, memory B cells, B-1 cells, B-2 cells, marginal-zone B cells, follicular B cells, germinal center B cells, or regulatory B cells (Bregs).

The present invention encompasses immune effector cells (e.g., T cells, NK cells, CIK cells, macrophages) comprising a recombinant nucleic acid construct comprising sequences encoding a CAR, e.g., a CAR molecule that binds to a tumor antigen (e.g., a TA CAR) or a CAR molecule that binds to a malignant B cell antigen (e.g., a BCA CAR), wherein the CAR comprises an antigen binding domain (e.g., antibody or antibody fragment) that binds specifically to a tumor antigen or a malignant B cell antigen.

Preferably, the antigen binding domain of the immune effector cells, e.g., a CAR molecule expressed by T cells, NK cells, CIK cells, or macrophages targets (e.g., binds to) a tumor antigen that is associated with a B cell malignancy, e.g., expressed by a B cell malignancy, preferably a hematological cancer.

In preferred embodiments, the tumor antigen that is targeted by the immune effector cells is present in a hematological cancer chosen from a leukemia or a lymphoma. Preferably, leukemias include, but are not limited to e.g., B-cell Acute Lymphoid Leukemia (“B-ALL”), T-cell Acute Lymphoid Leukemia (“T-ALL”), Acute Lymphoblastic Leukemia (ALL), Chronic Myelogenous Leukemia (CML) or Chronic Lymphoid Leukemia (CLL). Preferably, the leukemia is a relapsed or refractory B-cell precursor Acute Lymphoblastic Leukemia. Preferably, lymphomas include, but are not limited to Hodgkin's Disease, Non-Hodgkin's Lymphoma, Large B-Cell Lymphoma (LBCL), Diffuse Large B-Cell Lymphoma (DLBCL), primary mediastinal large B-cell lymphoma, high grade B-cell lymphoma and DLBCL arising from follicular lymphoma. Preferably, the lymphoma is a relapsed or refactory B-cell lymphoma.

The present invention provides CARs that can target the following exemplary B cell antigens including but not limited to: CD10, CD19, CD20, CD21, CD22, CD23, CD24, CD25, CD34, CD37, CD38, CD53, CD72, CD73, CD74, CD75, CD77, CD79a, CD79b, CD80, CD81, CD82, CD83, CD84, CD85, CD86, CD123, CD179b, ROR1, BCMA, and FLT3.

In a preferred embodiment, the CAR targets (e.g., binds to) CD19. More preferably, the immune effector cells that are engineered to contain one or more CARs that direct the immune effector cells to a malignant B cell are CD19-directed genetically modified autologous T-cells (e.g., tisagenlecluecel, axicabtagene ciloleucel) or CIK cells.

Antibodies that Target Malignant B Cells

The antibodies that target (e.g., bind to) malignant B cells target a tumor antigen that is associated with a B cell malignancy, e.g., expressed by a B cell malignancy, preferably a hematological cancer.

In preferred embodiments, the tumor antigen that is targeted by the antibodies is present in a hematological cancer chosen from a leukemia or a lymphoma. Preferably, leukemias include, but are not limited to e.g., B-cell Acute Lymphoid Leukemia (“B-ALL”), T-cell Acute Lymphoid Leukemia (“T-ALL”), Acute Lymphoblastic Leukemia (ALL), Chronic Myelogenous Leukemia (CML) or Chronic Lymphoid Leukemia (CLL). Preferably, the leukemia is a relapsed or refractory B-cell precursor Acute Lymphoblastic Leukemia. Preferably, lymphomas include, but are not limited to Hodgkin's Disease, Non-Hodgkin's Lymphoma, Large B-Cell Lymphoma (LBCL), Diffuse Large B-Cell Lymphoma (DLBCL), primary mediastinal large B-cell lymphoma, high grade B-cell lymphoma and DLBCL arising from follicular lymphoma. Preferably, the lymphoma is a relapsed or refactory B-cell lymphoma.

In a preferred embodiment, the antibody that targets malignant B cells targets a B cell antigen described herein, including but not limited to, CD19, CD20, CD22, CD123, FLT-3, ROR-1, CD79a, CD79b, CD179b, CD10, or CD34.

Examples of antibodies that target malignant B cells include monoclonal, polyclonal, bispecific antibodies, antibody conjugates (e.g., antibody-drug conjugates), or fragments thereof that target an antigen expressed on a malignant B cell, e.g., a malignant B cell antigen described herein, e.g., CD19, CD20, CD22, CD52, CD123, FLT-3, ROR-1, CD79a, CD79b, CD179b, CD10, or CD34. Preferably the antibodies that target malignant B cells include blinatumomab, rituximab, ofatumumab, ocrelizumab, veltuzumab, obinutuzumab, moxetumomab pasudotox, TRU-015, AME133V, Pro131921ibritumomab tiuxetan, tositumumab

In a preferred embodiment, the antibody that targets malignant B cells targets (e.g., binds to) CD22. For example, in an embodiment the antibody that targets malignant B cells that targets CD22 includes: an anti-CD22 monoclonal antibody-MMAE conjugate (e.g., DCDT2980S); an scFv of an anti-CD22 antibody, e.g., an scFv of antibody RFB4; an scFv of an anti-CD22 antibody fused to all of or a fragment of Pseudomonas exotoxin-A (e.g., BL22); a humanized anti-CD22 monoclonal antibody (e.g., epratuzumab); the Fv portion of an anti-CD22 antibody, which is optionally covalently fused to all or a fragment or (e.g., a 38 KDa fragment of) Pseudomonas exotoxin-A (e.g., moxetumomab pasudotox); or an anti-CD19/CD22 bispecific antibody, optionally conjugated or linked to a toxin such as a deglycosylated ricin A chain. Preferably, the antibody that targets malignant B cells is conjugated or otherwise bound to a cytotoxic agent or a chemotherapeutic agent. Preferably the antibody that targets malignant B cells is conjugated or otherwise bound to a cytotoxic agent (e.g., calicheamicins, ozogamicin). Preferably the antibody that targets malignant B cells is a CD22-directed antibody-drug conjugate comprising a recombinant humanized immunoglobulin antibody specific for human CD22 (e.g., inotuzumab), a calicheamicin and a linker that attaches the calicheamicin to the inotuzumab. Most preferably the CD22-directed antibody-drug conjugate that targets malignant B cells is moxetumomab pasudox (Lumoxiti®) and inotuzumab ozogamicin (e.g., BESPONSA® (inotuzumab ozogamicin) for Injection).

### Anti-Cancer Therapy

In an embodiment, the CAR immunotherapy as described herein is administered to the subject before, during, simultaneously with or after, administration of the antibody that targets malignant B cells. Preferably, the CAR immunotherapy is administered to the subject before or after administration of the antibody that targets malignant B cells. Most preferably, the CAR immunotherapy is administered to the subject before administration of the antibody that targets malignant B cells.

The immune effector cells (e.g., T cells, NK cells, CIK cells, macrophages) that are engineered to express a CAR targeting malignant B cells and/or the antibody that targets malignant B cells are administered to the subject using methods known in the art. Preferably, the immune effector cells and/or the antibody are administered parenterally, e.g., subcutaneously, intraperitoneally, intramuscularly or intravenously.

In some preferred embodiments, the subject is pre-medicated with acetaminophen and an H-1 antihistamine prior to administration of the immune effector cells (e.g., T cells, NK cells, CIK cells, macrophages) that are engineered to express a CAR targeting malignant B cells.

In some preferred embodiments, the subject is pre-medicated with a corticosteroid, antipyretic and antihistamine prior to administration of the antibody that targets malignant B cells.

In some embodiments, the immune effector cells (e.g., T cells, NK cells, CIK cells, macrophages) that are engineered to express a CAR targeting malignant B cells are administered intravenously, e.g., as an intravenous infusion. For example, each infusion provides about 104 to 109 cells/kg body weight, in some instances 105 to 106 cells/kg body weight, including all integer values within those ranges. Immune effector cell compositions may also be administered multiple times at these dosages.

In some embodiments, the antibody that targets malignant B cells is administered intravenously, e.g., as an intravenous infusion. For example, each infusion provides about 0.1-2000 mg of the antibody that targets malignant B cells, including all integer values within this range. In some embodiments, the antibody that targets malignant B cells is administered at a dose of 0.01 mg/m² to 750 mg/m, including all integer values within this range. Preferably, each infusion provides about 0.5-1 mg/m² 0.8-10 mg/m², 10-100 mg/m², 150-175 mg/m², 175-200 mg/m², 200-225 mg/m², 225-250 mg/m², 250-300 mg/m², 300-325 mg/m², 325-350 mg/m², 350-375 mg/m², 375-400 mg/m², 400-425 mg/m², 425-450 ng/n², 450-475 mg/m², 475-500 mg/m, 500-525 mg/m², 525-550 mg/m², 550-575 mg/m, 575-600 mg/m², 600-625 mg/m², 625-650 mg/m², 650-675 mg/m², or 675-700 mg/m², where m² indicates the body surface area of the subject. In some embodiments, the antibody that targets malignant B cells is administered at a dosing interval of at least 4 days, e.g., 4, 7, 14, 21, 28, 35 days, or more. For example, the antibody that targets malignant B cells is administered at a dosing interval of at least 0.5 weeks, e.g., 05, 1, 2, 3, 4, 5, 6, 7, 8 weeks, or more. In some embodiments, the antibody that targets malignant B cells is administered at a dose and dosing interval described herein for a period of time, e.g., at least 2 weeks, e.g., at least 2, 3, 4, 5, 6, 7, 8, 9, 10, 11, 12, 13, 14, 15, 16, 17, 18, 19, 20 weeks, or greater. For example, the antibody that targets malignant B cells is administered at a dose and dosing interval described herein for a total of at least 2 doses per treatment cycle (e.g., at least 2, 3, 4, 5, 6, 7, 8, 9, 10, 11, 12, 13, 14, 15, 16, or more doses per treatment cycle). Preferably, the antibody that targets malignant B cells is inotuzumab ozogamicin dosed according to the following dosing regimes for Cycle 1 and subsequent cycles depending on the response to treatment:

| | | |
|---|---|---|
| Day 1 | Day 8 | Day 15 |

Dosing regimen for Cycle 1

| All patients: | | | |
|---|---|---|---|
| Dose | 0.8 mg/m² | 0.5 mg/m² | 0.5 mg/m² |
| Cycle length | 21 days^{a} | | |
| Dosing regimen for subsequent cycles depending | | | |
| on response to treatment | | | |
| Patients who have achieved a CR or CRi: | | | |
| Dose | 0.5 mg/m² | 0.5 mg/m² | 0.5 mg/m² |

| | |
|---|---|
| Cycle length | 28 days |

Patients who have not achieved a CR or CRi:

| | | | |
|---|---|---|---|
| Dose | 0.8 mg/m² | 0.5 mg/m² | 0.5 mg/m² |

| | |
|---|---|
| Cycle length | 28 days |

^{a}For patients who achieve a CR or a CRi, and/or to allow for recovery from toxicity, the cycle length may be extended up to 28 days (i.e., 7-day treatment-free interval starting on Day 21).

The cells expressing chimeric antigen receptors (CARs) that target malignant B cells can be administered before (i.e., prior to), during (i.e., at the same time) or after (i.e, subsequent to) administration of the antibodies that target malignant B cells. In a preferred embodiment, the cells expressing chimeric antigen receptors (CARs) that target malignant B cells are administered prior to administration of the antibodies that target malignant B cells. In one preferred embodiment, the cells expressing chimeric antigen receptors (CARs) that target malignant B cells are administered 5 minutes, 10 minutes, 20 minutes, 30 minutes, 40 minutes, 50 minutes, 1 hour, 2 hours, 3 hours, 4 hours, 5 hours, 6 hours, 8 hours, 10 hours, 12 hours, 16 hours, 18 hours, 20 hours, 24 hours, 2 days, 3 days, 4 days, 5 days, 6 days, 7 days, 8 days, 9 days, 10 days, 11 days, 14 days 28 days, or more, prior to administration of the antibodies that target malignant B cells. Most preferably, the cells expressing chimeric antigen receptors (CARs) that target malignant B cells are administered 28 days prior to administration of the antibodies that target malignant B cells.

Doses of the immune effector cells (e.g., T cells, NK cells, CIK cells, macrophages) that are engineered to express a CAR targeting malignant B cells and/or the antibody that targets malignant B cells may be administered once, or more than once. In some embodiments, it is preferred that the immune effector cells are administered once (i.e., as a single administration) and the antibody is administered once a week, twice a week, three times a week, four times a week, five times a week, six times a week, or seven times a week for a predetermined duration of time. The predetermined duration of time may be 1 week, 2 weeks, 3 weeks, 4 weeks, 5 weeks, 6 weeks, 7 weeks, 2 months, 3 months, 4 months, 5 months, 6 months, 7 months, 8 months, 9 months, 10 months, 11 months, or up to 1 year or more.

In one embodiment, the methods of increasing or enhancing the efficacy of a CAR immunotherapy or the methods of treating a cancer (e.g., a B cell malignancy) comprise inhibiting the proliferation or reducing the population of cancer cells expressing a tumor antigen described herein, the methods comprising contacting a tumor antigen-expressing cancer cell population (e.g., a B cell malignancy) described herein with immune effector cells (e.g., T cells, NK cells, CIK cells, macrophages) that are engineered to express a CAR targeting malignant B cells in combination with an antibody that binds to a tumor antigen-expressing B cell described herein. In certain embodiments, the combination of the invention reduces the quantity, number, amount or percentage of cells and/or cancer cells by at least 25%, at least 30%, at least 40%, at least 50%, at least 65%, at least 75%, at least 85%, at least 95%, or at least 99% in a subject with a cancer associated with the expression of a tumor antigen as described herein, relative to a negative control. In one aspect, the cancer is a B cell malignancy, e.g., a hematologic cancer.

In preferred embodiments, the hematological cancer to be treated is chosen from a leukemia or a lymphoma. Preferably, leukemias include, but are not limited to e.g., B-cell Acute Lymphoid Leukemia (“B-ALL”), T-cell Acute Lymphoid Leukemia (“T-ALL”), Acute Lymphoblastic Leukemia (ALL), Chronic Myelogenous Leukemia (CML) or Chronic Lymphoid Leukemia (CLL). Preferably, the leukemia is a relapsed or refractory B-cell precursor Acute Lymphoblastic Leukemia. Preferably, lymphomas include, but are not limited to Large B-Cell Lymphoma (LBCL), Diffuse Large B-Cell Lymphoma (DLBCL), primary mediastinal large B-cell lymphoma, high grade B-cell lymphoma and DLBCL arising from follicular lymphoma. Preferably, the lymphoma is a relapsed or refactory B-cell lymphoma.

In one embodiment, the cells expressing a CAR molecule, e.g., a CAR molecule that targets malignant B cells described herein, are administered as a single, low dose which is not expected to provide any clinical benefit to the subject and the antibody that targets malignant B cells is administered at a dose that is not expected to result in complete remission (CR) of the cancer due to a high tumor burden at the time of dosing and/or poor physical condition of the subject.

The immune effector cells expressing a CAR molecule that targets B cells and the antibody that targets malignant B cells can target the same B cell antigen described herein or can target different B cell antigens described herein when used in combination. For example, the immune effector cells expressing a CAR molecule that targets a CD19 malignant B cell antigen can be used in combination with an antibody that targets a CD 19 malignant B cell antigen or with an antibody that targets a CD22 malignant B cell antigen.

In another embodiment, administration of an antibody that targets malignant B cells results in increased or prolonged proliferation of the CAR-expressing cells in a subject, e.g., as compared to a non-treated subject. In embodiments, increased proliferation is associated with in an increase in the number of the CAR-expressing cells. In another embodiment, administration of an antibody that targets malignant B cells results in increased killing of cancer cells (e.g., malignant B cells) by the CAR-expressing cells in a subject, e.g., as compared to a non-treated subject.

In another embodiment, the subjects receive an infusion of the CAR-expressing cells and the antibody that targets malignant B cells described herein prior to transplantation, e.g., allogeneic stem cell transplant, of cells.

The dosages and treatment schedules of the above treatments to be administered to a patient will vary with the precise nature of the condition being treated and the recipient of the treatment. The scaling of dosages for human administration can be performed according to art-accepted practices.

### Combination Therapies

The CAR-expressing cells that target malignant B cells in combination with the antibodies that target malignant B cells described herein may be used in further combination with other known agents and therapies.

The combination therapy described herein, e.g., immune effector cells (e.g., T cells, NK cells, CIK cells, macrophages) that are engineered to express one or more CARs targeting malignant B cells as described herein in combination with an antibody that binds to malignant B cells as described herein, can be administered in combination with at least one additional therapeutic agent. In an embodiment, the at least one additional therapeutic agent can be administered before, simultaneously or after the combination therapy described herein, in the same or in separate compositions, or sequentially. For sequential administration, the CAR-expressing cell described herein and/or the antibody that targets B cells, can be administered first, and the additional therapeutic agent can be administered second, or the order of administration can be reversed.

In another aspect of the present invention, kits that include one or more of the CAR-expressing cells that target malignant B cells and antibodies that target malignant B cells as disclosed herein are provided, whereby such kit may comprise a package insert or other labeling including directions for administration.

### Pharmaceutical Compositions

Pharmaceutical compositions of the present invention may comprise CAR-expressing cells, e.g., a plurality of CAR-expressing cells that target malignant B cells, as described herein, and/or antibodies that target malignant B cells, as described herein, in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose, dextrose or dextrans, mannitol; proteins; polypeptides or amino acids such as glycine; antioxidants; chelating agents such as EDTA or glutathione; adjuvants (e.g., aluminum hydroxide); human serum albumin, electrolytes (e.g., Plasma-Lyte A); and preservatives (e.g., Cryoserv® dimethylsulfoxide). Compositions of the present invention are in one aspect formulated for intravenous administration. Preferably, the CAR expressing cells that target malignant B cells are suspended in a patient-specific infusion bag and the antibody that targets malignant B cells is in the form of a lyophilized powder for reconstitution.

### EXAMPLE

The invention is further described in detail by reference to the following experimental example. This example is provided for purposes of illustration only and are not intended to be limiting unless otherwise specified. Thus, the invention should in no way be construed as being limited to the following example, but rather, should be construed to encompass any and all variations which become evident as a result of the teaching provided herein.

### Example 1

The unexpected clinical result regards a male adult patient, age 27, diagnosed with acute lymphoblastic leukemia (ALL), who had undergone ten rounds of standard of care therapy from which the patient had relapsed. This patient was subsequently qualified for enrollment into an experimental clinical phase-1/2a dose-escalating trial, studying the safety and efficacy of a single dose of CAR-modified T cells, designed to target the CD19 antigen, which is overexpressed on ALL cells and other B cell malignancies. The specific population of CAR-modified T cells used in this clinical trial is also known as a population of cytokine induced killer (CIK) cells, as defined by the concurrent expression of CD56 in a subpopulation of these T cells.

This patient was dosed with 1 million CAR-expressing T cells per kg body weight, which constitutes the lowest dose in this first-ever clinical trial, per regulatory approval. This dose was not expected to provide any clinical benefit to the patient, but would establish a first safety signal in humans. At the time of dosing with the CAR-modified T cells, the patient's bone marrow consisted of 60% blasts, which is considered to be a high amount of tumor burden, relative to the administered dose of CAR-expressing T cells.

The presence of the CAR-modified T cells in the patient's peripheral blood was monitored through measurement of vector copy numbers (VCN) using standard PCR methods. Typically, therapeutic activity of CAR-modified T cells corresponds with proliferation and expansion of these T cells in the patient's peripheral blood, which coincides with increasing VCN levels. By Day 14 following the infusion of the CAR-modified T cells, the VCN count in this patient had peaked to a 4645 copies/mcg fold increase compared to Day 0. At Day 21 and day 28, the VCN count was reduced respectively to 221 copies/mcg and below level of quantification, and at day 28 the patient's blast count in the bone marrow had increased to 90%. Given these observations, the dose of CAR-modified T cells was not considered to have provided any clinical benefit to this patient. As this patient's tumor burden continued to expand, he received a single administration of 0.8 mg/m² inotuzumab on Day 28, with the intent to control the rate of tumor growth.

Inotuzumab's therapeutic mechanism of action is able to facilitate tumor cell death. However, the administered dose was not expected to result in a complete remission (CR), because of the high tumor burden at the time of dosing and the poor physical condition of the patient, which is believed to have a negative impact on the therapeutic efficacy of inotuzumab. On Day +1 after inotuzumab administration, this patient suffered from a significant cytokine release syndrome (CRS), which required admission to the intensive care unit (ICU).

CRS is not typically observed in patients receiving stand-alone inotuzumab therapy (see inotuzumab Package Insert). Instead, CRS is a clinical adverse event seen in correlation with CAR-modified T cell therapy, especially in patients carrying high tumor burden. Therefore, the life-threatening CRS in this patient was an unexpected severe adverse event (SAE), also because of the perceived sub-therapeutic precedent dose of CAR-modified T cells and the absence of observed therapeutic effect of these cells, as measured by VCN and by the expanding tumor burden during the first 28 days after infusion of the CAR-modified T cells.

At Day +35, this patient was considered to have been in molecular CR, and remained stable in this condition for a subsequent 56 days, until he underwent an allogeneic bone marrow transplantation, with curative potential. At month 9 after infusion and month 5 after HSCT, the patient was still in molecular CR. Similar to the observed CRS, the clinical outcome of molecular CR was unexpected. On the other hand, molecular CR is often seen in patients treated with CAR-modified T cells as a stand-alone therapy, such as tisagenlecleucel, and is typically observed in conjunction with or following occurrence of CRS, where the intensity of CRS requires ICU admission of the patient.

The above described unexpected results support the hypothesis of a synergistic effect between inotuzumab and CAR-modified T cells, as these cells have shown the capacity to persist in the patients' peripheral blood for up to 70 days or longer. In this patient, inotuzumab was administered on Day 28 following infusion of the CAR-modified T cells, meaning that these cells could have been impacted by the administration of inotuzumab, thereby rendering them therapeutically active against the tumor cells.

The present invention relates to the design and preparation of polymeric hybrid core-shell nanocarriers of which the core is designed to bind transposons, transposases and/or plasmids and minicircles comprising transposon and/or transposases and the shell is designed to protect the payload, stabilize the nanocarrier, provide biocompatibility to the system, enable targeting to specific cells and tissue and promote efficient intracellular release of the payload from the nanocarrier.

I/We claim:

1. A method of increasing or enhancing the efficacy of a CAR immunotherapy cancer treatment that targets malignant B cells in a subject in need thereof comprising administering to the subject an antibody that targets malignant B cells in combination with the CAR immunotherapy.

2. The method of claim 1 wherein the cancer is a hematological cancer.

3. The method of claim 1 wherein the CAR immunotherapy comprises immune effector cells (e.g., T cells, NK cells, CIK cells, macrophages) engineered to express a BCA CAR.

4. The method of claim 3 wherein the immune effector cells target CD19.

5. The method of claim 1 wherein the antibody that targets B cells is a humanized or human monoclonal antibody.

6. The method of claim 5 wherein the antibody is inotuzumab ozogamicin.

7. A method of treating a B cell malignancy in a subject in need thereof comprising administering to the subject an antibody that targets malignant B cells in combination with a CAR immunotherapy cancer treatment that targets malignant B cells.

8. The method of claim 7 wherein the cancer is a hematological cancer.

9. The method of claim 7 wherein the CAR immunotherapy comprises immune effector cells (e.g., T cells, NK cells, CIK cells) engineered to express a BCA CAR.

10. The method of claim 9 wherein the immune effector cells target CD19.

11. The method of claim 7 wherein the antibody that targets B cells is a humanized or human monoclonal antibody.

12. The method of claim 11 wherein the antibody is inotuzumab ozogamicin.

13. The methods of claim 1 or 7 wherein the cancer is selected from leukemias and lymphomas.

14. The methods of any of the above claims except claim 13 wherein the CAR immunotherapy cancer treatment comprises administration of a single, low dose of the immune effector cells which is not expected to provide any clinical benefit to the subject and the antibody is administered at a dose that is not expected to result in complete remission (CR) of the cancer due to a high tumor burden at the time of dosing and/or poor physical condition of the subject.

15. The methods of claim 13 wherein the leukemias are selected from B-cell Acute Lymphoid Leukemia (“B-ALL”), T-cell Acute Lymphoid Leukemia (“T-ALL”), Acute Lymphoblastic Leukemia (ALL), Chronic Myelogenous Leukemia (CML) and Chronic Lymphoid Leukemia (CLL).

15. The methods of claim 13 wherein the lymphomas are selected from Hodgkin's Disease, Non-Hodgkin's Lymphoma, Large B-Cell Lymphoma (LBCL), Diffuse Large B-Cell Lymphoma (DLBCL), primary mediastinal large B-cell lymphoma, high grade B-cell lymphoma and DLBCL arising from follicular lymphoma.

16. The methods of claim 13 wherein the CAR immunotherapy cancer treatment comprises administration of a single, low dose of the immune effector cells which is not expected to provide any clinical benefit to the subject and the antibody is administered at a dose that is not expected to result in complete remission (CR) of the cancer due to a high tumor burden at the time of dosing and/or poor physical condition of the subject.
